SEQUENCE LISTING (21 sequences)

SEQ ID NO: 1 - protein, 494 aa, Artificial Sequence
  Composite H3 hemagglutinin with T-cell epitope (SEQ ID No 21)imposed
STATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSDNSFFSRLNWLTHLKFKYPALNVTMPNNEKFDKLYIWGVHHPGTDNDQISLYAQASGRITVSTKRSQQTVIPNIGSRPRVRDIPSRISIYWTIVKPGDMLLINSTGNLIAPRGYFKIRSGKSSIMRSDAPIGKCNSECITPNGSIPNDKPFQNVNRITYGACPKYVKQNTLKLATGMRNVPEKQTRGIFGAIAGFIENGWEGMVDGWYGFRHQNSEGIGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQI

SEQ ID NO: 2 - protein, 494 aa, Artificial Sequence
  Composite H3 hemagglutinin (SEQ ID NO 1) after one round of de-Antigenization
STATLCLGHHAVPNGTIVKTITSDQIEVTSATELVQSSSTGGICSSPHQILDGTNCTLIDALLGDPQCDGFQGKKWDLFVERSKAYSNCYPYDVPDYASLRSLVASSGTLEFTTESFNWTGVTQSGTSSACKRASTNSFFSRLNWLTHLKFKYPALNVTMPNNEKFDKLYIWGVHHPGTDNDQISLYAQASGRITVSTKRSQQTVIPNIGSRPRVRDIPSRISIYWTIVKPGDMLLINSTGNLIAPRGYFKIASGKSSIMTSSAPIGKCSSECITPGGSIPSSKPFQNVNRITYGACPKYVKQNTLKLATGMRNVPEKQTRGIFGAIAGFITNGWEGMVDGWYGFRHQNSEGIGQAADLKSTQAAIAQIAGKLNRLIGKTNTKFHQIEKAFSEVEGRIQDLEKYVEDTKIDLWSYNAELLVALENQHTIALTDSEMAKLFARTKKQLRENAEDMGNGCFKIYHKCDNACIGSIRNGTYDHDVYRDEALNNRFQI

SEQ ID NO: 3 - protein, 494 aa, Artificial Sequence
  Composite H3 hemagglutinin (SEQ ID NO 1) after two rounds of de-Antigenization
STATLCLGHHAVPNGTIVKTITSDQIEVTSATELVQSSSTGGICSSPHQILDGTNCTLIDALLGDPQCDGFQGKKWDLFVERSKAYSNCYPYDVPDYASLRSLVASSGTLEFTTESFSWTGVTQSGTSSACKRASTTSFFSRLNWLTHLKFKYPALTVTMPNSETFDKLYIWGVHHPGTAADQISLYAQASGRITVSTKASQQTVIPNIGSRPAVRGIPSRISIYWTIVKPGDMLLINSTGNLIAPRGYFKIASGKSSIMTSSAPIGKCSSECITPGGSIPSSKPFQNVNRITYGACPKYVKQNTLKLATGMRNVPEKQTRGIFGAIAGFITNGWEGMVDGWYGFRHQNSTGIGQAADLKSTQAAIAQIAGKLNRLIGKTNTKFHQIEKAFSEVEGRIQDLEKYVEDTKIDLWSYNAELLVALENQHTIALTDSEMAKLFARTKKQLRENAEDMGNGCFKIYHKCDAACIGSIRGGTYSHAVYRAEALANRFQI

SEQ ID NO: 4 - protein, 494 aa, Artificial Sequence
  Composite H3 hemagglutinin (SEQ ID NO 1) after three rounds of de-Antigenization
STATLCLGHHAVPNGTIVKTITSDQIEVTSATELVQSSSTGGICSSPHQILTGTGCTLIDALLGDPQCAGFQGKKWDLFVERSKAYSNCYPYDVPGYASLRSLVASSGTLAFTTESFSWTGVTQSGTSSACKRASTTSFFSRLNWLTHLKFKYPALTVTMPNSETFDKLYIWGVHHPGTAADQISLYAQASGRITVSTKASQQTVIPNIGSRPAVRGIPSRISIYWTIVKPGDMLLINSTGNLIAPRGYFKIASGKSSIMTSSAPIGKCSSECITPGGSIPSSKPFQNVNRITYGACPKYVKQNTLKLATGMRNVPEKQTRGIFGAIAGFITNGWEGMVDGWYGFRHQNSTGIGQAADLKSTQAAIAQIAGKLNRLIGKTNTKFHQIEKAFSTVEGRIQDLEKYVEDTKIDLWSYNAELLVALENQHTIALTDSEMAKLFARTKKQLRENAEDMGNGCFKIYHKCDAACIGSIRGGTYSHAVYRAEALANRFQI

SEQ ID NO: 5 - protein, 494 aa, Artificial Sequence
  Composite H3 hemagglutinin (SEQ ID NO 1) after four rounds of de-Antigenization
STATLCLGHHAVPNGTIVKTITSDQIEVTSATELVQSSSTGGICSSPHAILTGTGCTLIDALLGDPQCAGFQGKKWDLFVERSKAYSNCYPYDVPGYASLRSLVASSGTLAFTTESFSWTGVTQSGTSSACKRASTTSFFSRLNWLTHLKFKYPALTVTMPNSETFDKLYIWGVHHPGTAADQISLYAQASGRITVSTKASQQTVIPNIGSRPAVRGIPSRISIYWTIVKPGDMLLINSTGNLIAPRGYFKIASGKSSIMTSSAPIGTCSSTCITPGGSIPSSKPFQNVNRITYGACPKYVKQNTLKLATGMRNVPEKQTRGIFGAIAGFITNGWEGMVDGWYGFRHQNSTGIGQAADLKSTQAAIAQIAGKLNRLIGKTNTKFHQIEKAFSTVEGRIQDLEAYVEDTKIDLWSYNAELLVALENQHTIALTDSEMAKLFARTKKQLRENAEDMGNGCFKIYHKCDAACIGSIRGGTYSHAVYRAEALANRFQI

SEQ ID NO: 6 - protein, 388 aa, Influenza A virus
  PEPTIDE (1)..(388)
  PEPTIDE (1)..(388): Corresponds to pronase-released fragment of an N2 neuraminidase
AEYRNWSKPQCDITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYPGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 7 - protein, 388 aa, Artificial Sequence
  N2 neuraminidase (SEQ ID NO 6) after one round of de-Antigenization
AAYRSWSKPQCSITGFAPFSKDNSIRLSAGGDIWVTREPYVSCDPGKCYQFALGQGTTLNNVHSGGTVTGATPYRTLLMNELGVPFHLGTKQVCIAWSSSSCHDGKAWLHVCVTGSDKNATASFIYGGRLVDSIVSWSKEILRTQESECVCIGGTCTVVMTDGSASGKADTKILFIEEGKIVHTSTLSGSAQHVEECSCYPTYPGVRCVCRDNWKGSNRPIVDINIKGYSIVSSYVCSGLVGDTPRKSDSSSSSTCLGPNSEEGGTGVKGWAFDDGNDVWMGRTISEKLRSGYETFKVIEGWSGPNSKLQINRQVIVDTGNRSGYSGIFSVEGKSCINRCFYVELIRGRKTETTVLWTSNSIVVFCGTSGTYGTGSWPDGASIALMPI

SEQ ID NO: 8 - protein, 388 aa, Artificial Sequence
  N2 neuraminidase (SEQ ID NO 6) after two rounds of de-Antigenization
AAYRSWSKPQCSITGFAPFSKDNSIRLSAGGDIWVTREPYVSCDPGKCYQFALGQGTTLNNVHSGGTVTGATPYRTLLMNELGVPFHLGTKQVCIAWSSSSCHDGKAWLHVCVTGSDKNATASFIYGGRLVDSIVSWSKEILRTQESECVCIGGTCTVVMTDGSASGKADTKILFIEEGKIVHTSTLSGSAQHVEECSCYPTYPGVRCVCRDNWKGSNRPIVDINIKGYSIVSSYVCSGLVGDTPRKSDSSSSSTCLGPNSEEGGTGVKGWAFDDGNDVWMGRTISETLRSGYETFKVITGWSGPGSKLQINRQVIVDTGNRSGYSGIFSVEGKSCINRCFYVELIRGRKTETTVLWTSNSIVVFCGTSGTYGTGSWPDGASIALMPI

SEQ ID NO: 9 - protein, 388 aa, Artificial Sequence
  N2 neuraminidase (SEQ ID NO 6) after three rounds of de-Antigenization
AAYRSWSKPQCSITGFAPFSKDNSIRLSAGGDIWVTREPYVSCDPGKCYQFALGQGTTLNNVHSGGTVTGATPYRTLLMNELGVPFHLGTKQVCIAWSSSSCHDGKAWLHVCVTGSDKNATASFIYGGRLVDSIVSWSKEILRTQESECVCIGGTCTVVMTDGSASGKADTKILFIEEGKIVHTSTLSGSAQHVEECSCYPTYPGVRCVCRDNWKGSNRPIVDINIKGYSIVSSYVCSGLVGDTPRKSDSSSSSTCLGPNSEEGGTGVKGWAFDDGNDVWMGRTISETLRSGYETFKVITGWSGPGSKLQITRQVIVDTGNRSGYSGIFSVEGTSCINRCFYVELIRGRKTETTVLWTSNSIVVFCGTSGTYGTGSWPDGASIALMPI

SEQ ID NO: 10 - protein, 388 aa, Artificial Sequence
  N2 neuraminidase (SEQ ID NO 6) after four rounds of de-Antigenization
AAYRSWSKPQCSITGFAPFSKDNSIRLSAGGDIWVTREPYVSCDPGKCYQFALGQGTTLGNVHSGGTVTGATPYRTLLMNELGVPFHLGTKQVCIAWSSSSCHDGKAWLHVCVTGSDKNATASFIYGGRLVDSIVSWSKEILRTQESECVCIGGTCTVVMTDGSASGKADTKILFIEEGKIVHTSTLSGSAQHVEECSCYPTYPGVRCVCRDNWKGSNRPIVDITITGYSIVSSYVCSGLVGDTPRKSDSSSSSTCLGPNSEEGGTGVKGWAFDDGGDVWMGRTISETLRSGYETFKVITGWSGPGSKLQITRQVIVDTGNRSGYSGIFSVEGTSCINRCFYVELIRGRKTETTVLWTSNSIVVFCGTSGTYGTGSWPDGASIALMPI

SEQ ID NO: 11 - protein, 505 aa, Artificial Sequence
  Composite H5 hemagglutinin with T-cell epitope (SEQ ID NO 21) imposed
DQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKQNTLKLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISG

SEQ ID NO: 12 - protein, 505 aa, Artificial Sequence
  Composite H5 hemagglutinin (SEQ ID NO 11) after one round of de-Antigenization
DQICIGYHANNSTAQVDTIMAKGVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKQNTLKLATGLRNSPQRERRRKKRGLFGAIAGFIEGGWQGMVDGWYGYHHSNTQGSGYAADKASTQKAIAGVTNKVASIIDKMNTQFEAVGREFNNLERRIENLNKKMEDGFLDVWTYNAELLVLMENERTLAFHDSNVKNLYDKVRLQLRDNAKELGNGCFEFYHKCDAACMASVRAGTYSYPQYSAAARLKRATISG

SEQ ID NO: 13 - protein, 505 aa, Artificial Sequence
  Composite H5 hemagglutinin (SEQ ID NO 11) after two rounds of de-Antigenization
DQICIGYHANNSTAQVDTIMAKGVTVTHAQDILEKTHSGKLCDLGGVKPLILAGCSVAGWLLGNPMCDTFINVPEWSYIVEKASPVNDLCYPGDFNDYEELKHLLSRITHFTKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSALAYGTCTTKCQTPMGAISSSMPFHNIHPLTIGECPKYVKQNTLKLATGLRNSPQRERRRKKRGLFGAIAGFIEGGWQGMVDGWYGYHHSNTQGSGYAADKASTQKAIAGVTNKVASIIAKMSTQFEAVGREFNNLERRIENLNKKMEDGFLDVWTYNAELLVLMENERTLAFHDSNVKNLYDKVRLQLRDNAKELGNGCFEFYHKCDAACMASVAAGTYSYPQYSAAARLKRATISG

SEQ ID NO: 14 - protein, 505 aa, Artificial Sequence
  Composite H5 hemagglutinin (SEQ ID NO 11) after three rounds of de-Antigenization
DQICIGYHANNSTAQVDTIMAKGVTVTHAQDILEKTHSGKLCDLGGVKPLILAGCSVAGWLLGNPMCDTFINVPEWSYIVEKASPVNDLCYPGDFNGYEELKHLLSRITHFTKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSALAYGTCTTKCQTPMGAISSSMPFHNIHPLTIGECPKYVKQNTLKLATGLRNSPQRERRRKKRGLFGAIAGFIEGGWQGMVDGWYGYHHSNTQGSGYAADKASTQKAIAGVTNKVASIIAKMSTQFEAVGREFSGLERRIENLNKKMEDGFLDVWTYNAELLVLMENERTLAFHDSNVKNLYDKVRLQLRDNAKELGNGCFEFYHKCDAACMASVAAGTYSYPQYSAAARLKRATISG

SEQ ID NO: 15 - protein, 505 aa, Artificial Sequence
  Composite H5 hemagglutinin (SEQ ID NO 11) after four rounds of de-Antigenization
DQICIGYHANNSTAQVDTIMAKGVTVTHAQDILEKTHSGKLCDLGGVKPLILAGCSVAGWLLGNPMCDTFINVPEWSYIVEKASPVNDLCYPGDFNGYEELKHLLSRITHFTKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSALAYGTCTTKCQTPMGAISSSMPFHNIHPLTIGECPKYVKQNTLKLATGLRNSPQRERRRKKRGLFGAIAGFIEGGWQGMVDGWYGYHHSNTQGSGYAADKASTQKAIAGVTNKVASIIAKMSTQFEAVGREFSGLERRIENLNKKMEDGFLDVWTYNAELLVLMENERTLAFHDSNVKNLYDKVRLQLRDNAKELGNGCFEFYHTCDAACMASVAAGTYSYPQYSAAARLKRATISG

SEQ ID NO: 16 - protein, 386 aa, Influenza A virus
  PEPTIDE (1)..(386)
  PEPTIDE (1)..(386): Corresponds to pronase-released fragment of an N1 neuraminidase
SVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDSGAVAVLKYNGIITDTIKSWRNNILRTQESECACVNGSCFTVMTDGPSNGQASHKIFKMEKGKVVKSVELDAPNYHYEECSCYPDAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWIETDSSFSVKQDIVAITDWSGYSGSFVQHPELTGLDCIRPCFWVELIRGRPKESTIWTSGSSISFCGVNSDTVGWSWPDGAELPFTI

SEQ ID NO: 17 - protein, 386 aa, Artificial Sequence
  N1 neuraminidase (SEQ ID NO 16) after one round of de-Antigenization
SVKLAGNSSLCPIGGWAVYSKDNSIRIGSKGDVFVIREPFISCSHLECRTFFLTQGALLNDKHSNGTVKGRSPHRTLMSCPVGEAPSPYNSRFESVAWSASACHDGTSWLTIGISGPGSGAVAVLKYNGIITDTIKSWAGNILRTQESECACVNGSCFTVMTDGPSGGQASHKIFKMEKGKVVKSVELSAPNYHYEECSCYPDAGEITCVCRDNWHGSNRPWVSFNQNLEYQIGYICSGVFGDNPRPGSGTGSCGPVSSNGAYGVKGFSFKYGNGVWIGRTKSTGSRSGFEMIWDPNGWIATGSSFSVKQTIVAITDWSGYSGSFVQHPELTGLDCIRPCFWVELIRGRPKESTIWTSGSSISFCGVGSSTVGWSWPDGAELPFTI

SEQ ID NO: 18 - protein, 386 aa, Artificial Sequence
  N1 neuraminidase (SEQ ID NO 16) after two rounds of de-Antigenization
SVKLAGSSSLCPIGGWAVYSKDNSIRIGSKGDVFVIREPFISCSTLECRTFFLTQGALLNDKHSGGTVKGRSPHRTLMSCPVGEAPSPYNSTFESVAWSASACHDGTSWLTIGISGPGSGAVAVLKYNGIITDTIKSWAGNILRTQESECACVGGSCFTVMTDGPSGGQASHKIFKMEKGTVVKSVTLSAPNYHYEECSCYPDAGTITCVCRDNWHGSNRPWVSFNQGLAYTIGYICSGVFGDNPRPGSGTGSCGPVSSNGAYGVKGFSFKYGGGVWIGRTKSTGSRSGFEMIWDPNGWIATGSSFSVKQTIVAITDWSGYSGSFVQHPTLTGLGCIRPCFWVELIRGRPKESTIWTSGSSISFCGVGSSTVGWSWPDGAELPFTI

SEQ ID NO: 19 - protein, 386 aa, Artificial Sequence
  N1 neuraminidase (SEQ ID NO 16) after three rounds of de-Antigenization
SVKLAGSSSLCPIGGWAVYSKDNSIRIGSTGDVFVIREPFISCSTLECRTFFLTQGALLNDAHSGGTVKGASPHRTLMSCPVGEAPSPYNSTFESVAWSASACHDGTSWLTIGISGPGSGAVAVLKYNGIITDTIKSWAGGILRTQESECACVGGSCFTVMTDGPSGGAASHKIFKMEKGTVVKSVTLSAPNYHYEECSCYPDAGTITCVCRDNWHGSNRPWVSFNQGLAYTIGYICSGVFGDNPRPGSGTGSCGPVSSNGAAGVKGFSFKYGGGVWIGRTKSTGSRSGFEMIWDPNGWIATGSSFSVKQTIVAITDWSGYSGSFVQHPTLTGLGCIRPCFWVELIRGRPKESTIWTSGSSISFCGVGSSTVGWSWPDGAALPFTI

SEQ ID NO: 20 - protein, 386 aa, Artificial Sequence
  N1 neuraminidase (SEQ ID NO 16) after four rounds of de-Antigenization
SVKLAGSSSLCPIGGWAVYSKDNSIRIGSTGDVFVIREPFISCSTLTCRTFFLTQGALLGDAHSGGTVKGASPHRTLMSCPVGEAPSPYNSTFESVAWSASACHDGTSWLTIGISGPGSGAVAVLKYNGIITDTIKSWAGGILRTQESECACVGGSCFTVMTDGPSGGAASHKIFKMEKGTVVKSVTLSAPNAHYEECSCYPDAGTITCVCRDNWHGSNRPWVSFNQGLAYTIGYICSGVFGDNPRPGSGTGSCGPVSSNGAAGVKGFSFKYGGGVWIGRTKSTGSRSGFEMIWDPNGWIATGSSFSVKQTIVAITDWSGYSGSFVQHPTLTGLGCIRPCFWVELIRGRPKESTIWTSGSSISFCGVGSSTVGWSWPDGAALPFTI

SEQ ID NO: 21 - protein, 13 aa, Homo sapiens
  PEPTIDE (1)..(13)
  PEPTIDE (1)..(13): An immunodominant T-cell epitope
PKYVKQNTLKLAT

(19) United States
(12) Patent Application Publication
(10) Pub. No.: US 2009/0162383 A1
(43) Pub. Date: Jun. 25, 2009

(54) Method for designing vaccines against constantly mutating pathogens

(76) Inventor: Eduardo A. Padlan, Kensington, MD (US)

Correspondence Address: EDUARDO A. PADLAN, 4006 SIMMS DRIVE, KENSINGTON, MD, 20895, US

(21) Appl. No.: 11/645,448

(22) Filed: Dec. 26, 2006

Related U.S. Application Data
(60) Provisional application No. 60/755,870, filed on Jan. 4, 2006.

(51) Int. Cl. A61K 39/00; C07K 1/00; C07H 21/00; A61K 38/16; A61K 31/7088; A61P 31/00

(52) U.S. Cl. 424/184.1; 530/403; 536/23.1; 514/12; 514/44

(57) ABSTRACT

A unique method is disclosed for identifying and replacing surface amino acid residues of a protein antigen that reduces the antigenicity of the putative immunodominant epitopes of the antigen and makes all the accessible regions of the molecule essentially antigenically equivalent, so that the antibody response will be directed against more parts of the molecule and not mainly against the erstwhile immunodominant epitopes. The method will simultaneously change the antigenicity of the molecule and preserve its structure. The method is useful in the design of molecules useful for immunization, for example, as vaccines, or for the generation of therapeutic antibodies, against constantly mutating pathogens. It is also useful in the design of molecules useful for immunization against pathogens that had been intentionally mutated so as to render those pathogens able to infect erstwhile immune individuals.

### REFERENCES CITED

Alix, A. J., Predictive estimation of protein linear epitopes by using the program PEOPLE, 1999, Vaccine, 18, pp. 311-314.
Barbey-Martin, C. et al., An antibody that prevents the hemagglutinin low pH fusogenic transition, 2002, Virology. 294, pp. 70-74.
Batori, V. et al., An in silico method using an epitope motif database for predicting the location of antigenic determinants on proteins in a structural context, 2006, J. Mol. Recognit., 19, pp. 21-29.
Benjamin, D. C. et al., The antigenic structure of proteins: a reappraisal, 1984, Annu. Rev. Immunol., 2, pp. 67-101.
Berman, H. M. et al., The Protein Data Bank, 2000, Nuc. Acids Res., 28, pp. 235-242.
Bianchi, E. et al., Universal influenza B vaccine based on the maturational cleavage site of the hemagglutinin precursor, 2005, J. Virol., 79, pp. 7380-7388.
Bush, R. M. et al., Predicting the evolution of human influenza A, 1999, Science, 286, pp. 1921-1925.
Chen, J. et al., Structure of the hemagglutinin precursor cleavage site, a determinant of influenza pathogenicity and the origin of the labile conformation, 1998, Cell, 95, pp. 409-417.
Chi, X. S. et al., Molecular evolution of human influenza A/H3N2 virus in Asia and Europe from 2001 to 2003, 2005, J. Clin. Microbiol., 43, pp. 6130-6132.
Davies, D. R. et al., Antibody-antigen complexes, 1988, J. Biol. Chem., 263, pp. 10541-10544.
De Genst, E. et al., Kinetic and affinity predictions of a protein-protein interaction using multivariate experimental design, 2002, J. Biol. Chem., 277, pp. 29897-29907.
Fazekas de St. Groth, S. Antigenic, adaptive and adsorptive variants of the influenza haemagglutinin, 1977, In Topics in Infectious Diseases Vol. 3 (R. G. Laver, H. Bachmayer, and R. Weil, editors) (Springer-Verlag, Vienna), pp. 25-48.
Fleury, D. et al., Antigen distortion allows influenza virus to escape neutralization, 1998, Nat. Struct. Biol., 5, pp. 119-123.
Fleury, D. et al., A complex of influenza hemagglutinin with a neutralizing antibody that binds outside the virus receptor binding site, 1999, Nat. Struct. Biol., 6, pp. 530-534.
Fleury, D. et al., Structural evidence for recognition of a single epitope by two distinct antibodies, 2000, Proteins, 40, pp. 572-578.

Fynan, E. F. et al., DNA vaccines: protective immunizations by parenteral, mucosal, and gene-gun inoculations, 1993, Proc. Natl. Acad. Sci. U.S.A., 90, pp. 11478-11482.
Grantham, R., Amino acid difference formula to help explain protein evolution, 1974, Science, 185, pp. 862-864.
Guex, N. et al., SWISS-MODEL and the Swiss-PdbViewer: An environment for comparative protein modeling, 1997, Electrophoresis, 18, pp. 2714-2723.
Gulati, U. et al., Antibody epitopes on the neuraminidase of a recent H3N2 influenza virus (A/Memphis/31/98), 2002, J. Virol., 76, pp. 12274-12280.
Ha, Y. et al., H5 avian and H9 swine influenza virus haemagglutinin structures: possible origin of influenza subtypes, 2002, EMBO J., 21, pp. 865-875.
Hennecke, J. et al., Structure of a complex of the human alpha/beta T cell receptor (TCR) HA1.7, influenza hemagglutinin peptide, and major histocompatibility complex class II molecule, HLA-DR4 (DRA*0101 and DRB1*0401): insight into TCR cross-restriction and alloreactivity, 2002, J. Exp. Med., 195, pp. 571-581.
Hoffmann, E. et al., Role of specific hemagglutinin amino acids in the immunogenicity and protection of H5N1 influenza virus vaccines, 2005, Proc. Natl. Acad. Sci. U.S.A., 102, pp. 12915-12920.
Hopp, T. P. et al., Prediction of Protein Antigenic Determinants from Amino Acid Sequences, 1981, Proc. Natl. Acad. Scis. U.S.A., 78, pp. 3824-3828.
Hopp, T. P. et al., A computer program for predicting protein antigenic determinants, 1983, Mol. Immunol., 20, pp. 483-489.
Horimoto, T. et al., Reverse genetics provides direct evidence for a correlation of hemagglutinin cleavability and virulence of an avian influenza A virus, 1994, J. Virol., 68, pp. 3120-3128.
Horvath A, T. et al., A hemagglutinin-based multipeptide construct elicits enhanced protective immune response in mice against influenza A virus infection, 1998, Immunol. Lett. 60, pp. 127-136.
Huylebroeck, D. et al., Viral delivery systems for Heterologous Antigens and Epitopes, 1988, Technological Advances in Vaccine Development, 84, pp. 279-293.
Jameson, B. A. et al., The antigenic index: a novel algorithm for predicting antigenic determinants, 1988, Comput. Appl. Biosci., 4, pp. 181-186.
Kabsch, W. et al., Dictionary of protein secondary structure: pattern recognition of hydrogen-bonded and geometrical features, 1983, Biopolymers, 22, pp. 2577-2637.
Kaverin, N. V. et al., Structure of antigenic sites on the haemagglutinin molecule of H5 avian influenza virus and phenotypic variation of escape mutants, 2002, J. Gen. Virol., 83, pp. 2497-2505.
Kilbourn, E. D. et al., Independent and disparate evolution in nature of influenza A virus hemagglutinin and neuraminidase glycoproteins, 1990, Proc. Natl. Acad. Sci. U.S.A. 87, pp. 786-790.
Kulkarni-Kale, U. et al., CEP: a conformational epitope prediction server, 2005, Nuc. Acids Res., 33, pp. W168-W171.
Lee, M. S. et al., Predicting antigenic variants of influenza A/H3N2 viruses, 2004, Emerg. Infect. Dis., 10, pp. 1385-1390.
Macken, C. et al., The value of a database in surveillance and vaccine selection, 2001, In Options for the Control of Influenza IV. A. D. M. E. Osterhaus, N. Cox & A. W. Hampson (Eds.) Amsterdam: Elsevier Science, pp. 103-106.
Maksyutov, A. Z. et al, ADEPT: a computer program for prediction of protein antigenic determinants, 1993, Comput. Appl. Biosci., 9, pp. 291-297.
Malby, R. L. et al., The structure of a complex between the NC10 antibody and influenza virus neuraminidase and comparison with the overlapping binding site of the NC41 antibody, 1994, Structure, 2, pp. 733-746.
Martinet, A. et al., Evaluation of recombinant A/Victoria/3/75 (H3N2) influenza neuraminidase mutants as potential broad-spectrum subunit vaccines against influenza, 1998, Arch. Virol., 143, pp. 2011-2019.
Moore G. R. et al., Comparison of the structures of various eukaryotic ferricytochromes c and ferrocytochromes and their antigenic differences, 1980, Eur. J. Biochem., 103, pp. 543-550.
Nakajima, K. et al., Restriction of amino acid change in influenza A virus H3HA: comparison of amino acid changes observed in nature and in vitro, 2003, J. Virol., 77, pp. 10088-10098.
Novotny, J. et al, Antigenic determinants in proteins coincide with surface regions accessible to large probes (antibody domains), 1986, Proc. Natl. Acad. Sci. U.S.A., 83, pp. 226-230.
Novotny, J., Protein antigenicity: a thermodynamic approach, 1991, Mol. Immunol., 28, pp. 201-227.
Odorico, M. et al., BEPITOPE: predicting the location of continuous epitopes and patterns in proteins, 2003, J. Mol. Recognit., 16, pp. 20-22.
Pace, C. N. et al., A helix propensity scale based on experimental studies of peptides and proteins, 1998, Biophys. J., 75, pp. 422-427.
Padlan, E. A., Quantitation of the immunogenic potential of protein antigens, 1985, Mol. Immunol., 22, pp. 1243-1254.
Padlan. E. A. On the Nature of Antibody Combining Sites: Unusual Structural Features That May Confer on These Sites an Enhanced Capacity for Binding Ligands, 1990, PROTEINS: Struct. Funct. Genet., 7, pp. 112-124.
Padlan, E. A., Anatomy of the Antibody Molecule, 1994, Mol. Immunol., 31, pp. 169-217.
Padlan, E. A. X-ray Crystallography of Antibodies, 1996, Adv. Prot. Chem., 49, pp. 57-133.
Peitsch, M. C. Protein modeling by E-mail, 1995, Bio/Technology, 13, pp. 658-660.
Pellequer, J. L. et al., PREDITOP: a program for antigenicity prediction, 1993, J. Mol. Graph., 11, pp. 204-210.
Plotkin, J. B. et al., Codon bias and frequency-dependent selection on the hemagglutinin epitopes of influenza A virus, 2003, Proc. Natl. Acad. Sci. U.S.A., 100, pp. 7152-7157.
Puthavathana, P. et al., Molecular characterization of the complete genome of human influenza H5N1 virus isolates from Thailand, 2005, J. Gen. Virol., 86, pp. 423-433.
Rammensee, H.-G. et al., SYFPEITHI: database for MHC ligands and peptide motifs, 1999, Immunogenetics, 50, pp. 213-219.
Robinson, H. L. et al., Protection against a lethal influenza virus challenge by immunization with a haemagglutinin-expressing plasmid DNA, 1993, Vaccine, 11, pp. 957-960.
Rost, B. et al., Prediction of protein secondary structure at better than 70% accuracy, 1993, J. Mol. Biol., 232, pp. 584-599.
Saha, S. et al., BcePred: Prediction of continuous B-cell epitopes in antigenic sequences using physico-chemical properties, 2004, In ICARIS, LNCS (Nicosia G, Cutello V, Bentley P J, Timmis J., editors) Vol. 3239 (Springer, Berlin), pp. 197-204.
Saha, S. et al., Prediction of continuous B-cell epitopes in an antigen using recurrent neural network, 2006, Proteins, 65, pp. 40-48.
Sandberg, M. et al., New chemical descriptors relevant for the design of biologically active peptides. A multivariate characterization of 87 amino acids, 1998, J. Med. Chem., 41, pp. 2481-2491.
Schwede T. et al., SWISS-MODEL: an automated protein homology-modeling server, 2003, Nuc. Acids Res., 31, pp. 3381-3385.
Skehel, J. J. et al., Studies on the primary structure of the influenza virus hemagglutinin, 1975, Proc. Natl. Acad. Sci. U.S.A., 72, pp. 93-97.
Skehel, J. J. et al., Receptor binding and membrane fusion in virus entry: the influenza hemagglutinin, 2000, Annu. Rev. Biochem., 69, pp. 531-569.
Smith, B. J. et al., Analysis of inhibitor binding in influenza virus neuraminidase, 2001, Protein Sci., 10, pp. 689-696.
Smith, D. J. et al., Mapping the antigenic and genetic evolution of influenza virus, 2004, Science, 305, pp. 371-376.
Sneath, P. H., Relations between chemical structure and biological activity in peptides, 1966, J. Theor. Biol., 12, pp. 157-195.
Sollner, J. et al., Machine learning approaches for prediction of linear B-cell epitopes on proteins, 2006, J. Mol. Recognit., 19, pp. 200-208.
Street, A. G. et al., Intrinsic beta-sheet propensities result from van der Waals interactions between side chains and the local backbone, 1999, Proc. Natl. Acad. Sci. U.S.A., 96, pp. 9074-9076.
Temoltzin-Palacios, F. et al., Modulation of Immunodominant Sites in Influenza Hemagglutinin Compromise Antigenic Variation and Select Receptor-binding Variant Viruses, 1994, J. Exp. Med., 179, pp. 1719-1724.
Thornton, J. M. et al., Location of ‘continuous’ antigenic determinants in the protruding regions of proteins, 1986, EMBO J., 5, pp. 409-413.
Tulip, W. R. et al., Refined crystal structure of the influenza virus N9 neuraminidase-NC41 Fab complex, 1992, J. Mol. Biol., 227, pp. 122-148.
Varghese, J. N. et al., Three-dimensional structure of the neuraminidase of influenza virus A/TOKYO/3/67 at 2.2 Angstroms resolution, 1991, J. Mol. Biol., 221, pp. 473-486.
Varghese, J. N. et al., The three dimensional structure of the complex of 4-guanidino-neus6ac2en and influenza virus neuraminidase, 1995, Prot. Sci., 4, pp. 1081-1087.
Venkatramani, L. et al., An epidemiologically significant epitope of a 1998 human influenza virus neuraminidase forms a highly hydrated interface in the NA-antibody complex, 2006, J. Mol. Biol., 356, pp. 651-663.
Westhof, E. et al., Correlation between segmental mobility and the location of antigenic determinants in proteins, 1984, Nature, 311, pp. 123-126.
Wiley, D. C. et al., Structural identification of the antibody-binding sites of Hong Kong influenza haemagglutinin and their involvement in antigenic variation, 1981, Nature, 289, pp. 373-378.
Wilson, I. A. et al., Structure of the haemagglutinin membrane glycoprotein of influenza virus at 3 A resolution, 1981, Nature, 289, pp. 366-373.
Wilson, I. A. et al., Structural basis of immune recognition of influenza virus hemagglutinin, 1990, Annu. Rev. Immunol., 8, pp. 737-771.

### DESCRIPTION OF THE DRAWINGS

Table 1 shows the structural characteristics of the different naturally occurring amino acids and the amino-acid replacements designed to reduce the antigenicity of protein epitopes, hereinafter referred to as the process of de-Antigenization.

FIG. 1 shows the plots of antigenicity vs residue position for the H3 hemagglutinin, ISDN110519, before (top) and after four rounds of de-Antigenization (bottom).

FIG. 2 shows the plots of antigenicity vs residue position for the N2 neuraminidase, ISDN117767, before (top) and after four rounds of de-Antigenization (bottom).

FIG. 3 shows the plots of antigenicity vs residue position for the H5 hemagglutinin, AAS89004, before (top) and after four rounds of de-Antigenization (bottom).

FIG. 4 shows the plots of antigenicity vs residue position for the N1 neuraminidase, AAS89005, before (top) and after four rounds of de-Antigenization (bottom).

### FIELD OF THE INVENTION

This invention relates to the design of molecules useful for immunization, for example, as vaccines, or for the generation of therapeutic antibodies.

### BACKGROUND OF THE INVENTION

Many pathogens are able to evade the immune system by constantly mutating their surface molecules. In so doing, the antibodies, which had been generated against previous strains of the pathogens, are no longer totally protective. In many instances, the pathogens are able to make the evasive move, by mutating a few amino acids in a surface molecule that may contain several hundred residues.

Among the pathogens that are constantly mutating to achieve immune evasion are the influenza viruses, the cold virus, and the virus that causes AIDS. These and other viruses, as well as other pathogens, have molecules on their surface, some of which are used to recognize and bind to the cells that the pathogens infect. Those surface molecules are the targets of the antibodies that our immune system produces to combat the pathogens. The external regions of those molecules can be used as vaccines. But a vaccine that utilizes a surface molecule, or part thereof, of a particular pathogen will be effective against mainly that pathogen and may not offer protection against new, or other, strains of the pathogen.

The method described here is the judicious modification of the external regions of surface molecules of antigenically variable pathogens, so that the modified molecules may be useful for immunization, for example, as effective vaccines, or for the generation of therapeutic antibodies, against more and, potentially all, strains of the pathogens. The method can also be used to design molecules useful against pathogens that had been intentionally mutated so as to render those pathogens able to infect erstwhile immune individuals.

It is generally accepted that every accessible region of an antigen could elicit an antibody response (Benjamin et al., 1983; Davies et al., 1988). Nevertheless, it is recognized that certain regions are substantially more antigenic than others; these are the immunodominant epitopes.

Intuitively, it would seem obvious that constantly mutating pathogens could continually achieve immune evasion by having immunodominant epitopes on their surface molecules and localizing the mutations in those epitopes.

If we are able to identify the immunodominant epitopes and reduce the antigenicity of those epitopes, we might be able to divert the antibody response to more parts of the antigen and not mainly to its erstwhile immunodominant epitopes. The early results of Fazekas de St. Groth (1977) hinted at that possibility, which has been explored by others (e.g., Martinet et al., 1998; Temoltzin-Palacios et al., 1994).

The definitive identification of the residues which constitute an antigenic epitope can only be accomplished by a three-dimensional structural analysis of the antibody-antigen complex (e.g., Barbey-Martin et al., 2002; Davies et al., 1988; Fleury et al., 1998, 1999, 2000; Malby et al., 2004; Padlan, 1996; Tulip et al., 1992; Venkatramani et al., 2006; among others). Various procedures have been proposed to locate the continuous, as well as the linear, epitopes of proteins by computation (e.g., Alix, 1999; Batori et al., 2006; Hopp et al., 1981, 1983; Jameson et al., 1988; Kulkarni-Kale et al., 2005; Maksyutov et al., 1993; Novotny et al., 1986; Odorico et al., 2003; Padlan, 1985; Pellequer et al., 1993; Saha et al., 2004, 2006; Sollner et al., 2006; Thornton et al., 1986; Westhof et al., 1984; among others). Some residues in antigenic epitopes, but not all, could be identified by the analysis of natural, or laboratory-induced, mutants (e.g., Gulati et al., 2002; Kaverin et al., 2002; Nakajima et al., 2003; Hoffmann et al., 2005; among others). In some cases, the identity of residues in antigenic epitopes could be inferred from a comparison of the amino sequences of closely related strains of the pathogen (e.g., Bush et al., 1999; Chi et al., 2005; Kilbourne et al., 1990; Lee et al., 2004; Plotkin et al., 2002; Smith et al., 2004; among others), especially when the three-dimensional structure of the antigen is available.

The method described here locates, by purely computational means, the putative immunodominant epitopes of a protein antigen (putative because it is impossible to identify and delineate all the immunodominant epitopes of any antigen) and identifies the residues within those epitopes. The method also suggests how those residues should be replaced to reduce antigenicity. The method requires three-dimensional structural information for the antigen and makes use of the known differences in the physicochemical properties of the amino acids. Three-dimensional structural data for many proteins are available from the Protein Data Bank (Berman et al., 2000) and parameters describing the differences in the physicochemical properties of the naturally occurring amino acids have been calculated by various authors (e.g., Sneath, 1966; Grantham, 1974; Sandberg et al., 1998; among others). Those physicochemical parameters have been found useful in quantifying the reactivities and interaction of antibodies and antigens (e.g., Padlan, 1985, 1990, 1994; Novotny, 1991; among others) and other protein-protein interactions (e.g., De Genst et al., 2002; among others). The procedure for computing antigenicities in the method described here follows that used earlier (Padlan, 1985), with major differences. Those differences will be explained in detail below.

The method described here results in the amino acid sequences of de-Antigenized antigens that, in forms known to those skilled in the art, could be used as molecules useful for immunization, for example, as vaccines, or for the generation of therapeutic antibodies. Additionally, those amino acid sequences could serve as the basis for DNA versions of those molecules (e.g., Fynan et al., 1993; Huylebroeck et al., 1988; Robinson et al., 1993; among others).

### BRIEF SUMMARY OF THE INVENTION

The de-Antigenization of the putative immunodominant epitopes is achieved through the following steps:

(Step 1) Identify a protein molecule that has been identified as a major antigen in a particular pathogen.
(Step 2) Calculate the antigenicity of the various regions of the antigen, using three-dimensional structural information about the molecule and the known physicochemical properties of the amino-acid residues. Locate the regions with high antigenicities, i.e. the putative immunodominant epitopes.
(Step 3) Identify the amino-acid residues comprising the putative immunodominant epitopes, in particular those residues which, by virtue of their physicochemical properties and their accessibility, can contribute significantly to tight binding by antibody. Replace those residues with amino acids that would be expected to contribute less to the binding by antibody, while ensuring that the replacements will not significantly alter the structure of the antigen. At least one T-cell epitope should be preserved.
(Step 4) Using the new structure (the structure with the replacements), repeat Steps 2 and 3 as needed until the putative immunodominant epitopes have significantly lower antigenicities.
(Step 5) The amino acid sequences, which result in significantly lower antigenicities for the putative immunodominant epitopes, and polynucleotides derived from those sequences provide the basis for molecules useful for immunization, for example, as vaccines, or for the generation of therapeutic antibodies, against the pathogen.

### DETAILED DESCRIPTION OF THE INVENTION

Information about the three-dimensional structure of a particular antigen is often available from the Protein Data Bank (Berman et al., 2000) (http://www.rcsb.org/pdb). In the absence of experimentally-determined three-dimensional information, a model could be made based on structural information from closely related molecules. Various techniques are available for modeling purposes and those techniques are known to those skilled in the art.

On the basis of the three-dimensional structure of the antigen, the solvent accessibilities of the individual amino acid residues are computed using standard methods (see, for example, Padlan, 1990; Padlan 1994). Solvent accessibilities could also be obtained using the program DSSP (Kabsch et al., 1983) (implemented, for example, in http://bioweb.pasteur.fr/seqanal/interfaces/dssp-simple.html). The solvent accessibilities are used as weighting factors in the calculation of the antigenicities. The use of solvent accessibilities as weighting factors de-emphasizes the contribution of residues that are not too accessible and that probably do not contribute much to the interaction with antibody.

The method, which had been proposed earlier for quantifying the antigenicity of a given region in a protein molecule using the physicochemical attributes of the amino acid residues in the region (Padlan, 1985), is particularly suitable for locating the putative immunodominant epitopes and is followed here. Structural parameters describing the physicochemical attributes of the various amino acids have been computed by various authors (for example, Sneath, 1966; Grantham, 1974; Sandberg et al., 1998) and those can be used in the calculation of antigenicities. The antigenicity of a region in the molecule is computed by taking the sum of the structural parameters, weighted or unweighted, corresponding to all the residues within that region. Structural parameters have been shown to provide a good measure of the ability of a given region to participate in antibody-antigen and other protein-protein interactions (see, for example, Padlan, 1990; De Genst et al., 2002). Thus, antigenicity computed in this manner is directly correlated with the ability of a particular region to engage in tight binding to antibody. The regions displaying highest antigenicities are identified as the putative immunodominant epitopes.

One major difference between the method proposed earlier (Padlan, 1985) and the method described here is the size of the presumed antigenic epitope. In the earlier method, a radius of 8.5 Angstroms was used as the extent of the epitope. The value of 8.5 Angstroms was a guess, in view of the fact that no structure was available at that time for any antibody-antigen complex. The structures of many antibody-antigen complexes are now available in the Protein Data Bank. In the method described here, a more appropriate value of the radius is calculated from the known structures of the complexes of antibodies with the particular antigen. Another major difference between the earlier method and the one described here is the suggested use of the solvent accessibilities of the residues as weights in computing the contribution of those residues to the total antigenicity value; no weights were used in the earlier method. A further difference is the use here of the simple sum, as opposed to the use of the average in the earlier procedure, of the antigenicity contributions of the individual residues in the evaluation of the total antigenicity of the epitope.

The de-Antigenization of the putative immunodominant epitopes is achieved by the judicious replacement of the residues in those epitopes with amino acids that would contribute less to the total antigenicity values, while preserving the structure of the molecule. By taking into account the physicochemical properties of the amino acids and their propensity to participate in a particular secondary structure (presented in Table 1), replacement rules could be proposed. The replacement rules used in the examples below are included in Table 1. Other replacement rules could be proposed and used provided that they result in reduced antigenicity while preserving structure. The concept can be implemented by those skilled in the art using the following, or similar, algorithm:

(A.1.0)—Generate a set of amino-acid replacement rules based on structural criteria, e.g., the replacement rules in Table 1. The recommended structural criteria are (1) the replacing amino acid should contribute less to the binding interaction with an antibody and (2) the replacement should not result in a significant change in the structure of the molecule.
(A.2.0)—Identify a protein molecule that is a major antigen in a particular pathogen. Locate on the sequence the known T-cell epitopes of the molecule; if T-cell epitopes had not been experimentally determined, obtain possible T-cell epitopes using predictors, e.g. SYFPEITHI (Rammensee et al., 1999) (http://www.syfpeithi.de). If an experimentally-determined three-dimensional structure is available for the antigen, proceed to (A.3.0);
(A.2.1)—If a model structure for the antigen is available, proceed to (A.3.0);
(A.2.2)—Identify a homologous molecule for which an experimentally-determined three-dimensional structure or a model structure is available; if there is none, STOP
(A.2.3)—Generate a model for the antigen from its amino acid sequence.
(A.3.0)—Generate atomic coordinates for the biological, i.e. natural, aggregation state of the molecule (dimer, trimer, etc.) using appropriate symmetry operations. For experimentally-determined structures, atomic coordinates for the biological aggregation state may already be available from the Protein Data Bank. All subsequent computations should be on the biological aggregation state of the molecule.
(A.4.0)—Choose and isolate the positions at which the antigenicities will be computed, e.g., the alpha-carbon positions.
(A.5.0)—Compute the solvent accessibilities of the individual amino acid residues by using standard procedures (as described in Padlan, 1990 and references cited therein), or by using program DSSP (Kabsch et al., 1983) (implemented, for example, in http://bioweb.pasteur.fr/seqanal/interfaces/dssp-simple.html).
(A.6.0)—Choose a set of structural parameters (physicochemical attributes) for use in the computation of the antigenicities. The structural parameters compiled by Sandberg et al. (1998), or by Grantham (1974), are particularly suitable for the computation of antigenicities.
(A.7.0)—Compute the antigenicities at the positions chosen in (A.4.0). A measure of antigenicity ascribed to a given position would be the total contribution of the amino acids within a defined region around that position. The contribution of each amino acid may be the sum, appropriately weighted or unweighted, of the structural parameters chosen in (A.6.0). The solvent accessibility of the amino acid, computed in (A.5.0), is recommended as an appropriate overall weight for the contribution of that amino acid to the antigenicity.
(A.8.0)—Identify the possible location of the putative immunodominant epitopes. The positions with antigenicity values significantly higher than the rest are most probably part of the putative immunodominant epitopes. A basis for the identification of the putative immunodominant epitopes could be the root-mean-square (r.m.s.) deviation from the mean of the antigenicity values of all epitopes.
(A.9.0)—Replace the residues comprising the putative immunodominant epitopes according to the replacement rules generated in (A.1.0). The residues would be the ones located within a certain radius of the epitope centers chosen in (A.4.0). A suitable value for the radius could be determined by examining known antibody-antigen complexes (see, for example, Padlan, 1996). It is recommended that the residues to be replaced be chosen on the basis of their solvent accessibility and their relative contribution to the overall antigenicity of the epitope. Preserve those residues which are probably critical to the structure (secondary, tertiary, quaternary) of the antigen, including residues whose posttranslational modification, e.g. glycosylation, is probably required for preservation of structure. Preserve at least one of the T-cell epitopes located in (A.2.0), as well as segments for which high antigenicity values might elicit useful antibody responses, e.g. inhibition of particular reactions. The suggested replacement should not be made if it will result in a peptide segment (of sufficient length to be presented by T cells) that is identical to a segment present in a human protein; this is to obviate autoimmune reactions.
(A.10.0)—Repeat (A.2.3) to (A.9.0) until it is deemed that the decrease in antigenicity of the putative immunodominant epitopes is sufficient, or until no further amino-acid replacements are warranted.
(A.11.0)—The amino acid sequences resulting from (A.10.0), or the polynucleotides derived from those sequences, provide the basis for molecules useful for immunization, for example, as vaccines, or for the generation of therapeutic antibodies, against the pathogen.

The present invention will now be described with reference to the following specific, non-limiting examples.

The particular pathogen in these examples is the influenza virus of which there are three types: A, B, and C, and the target antigens are the two surface glycoprotein molecules on the virus: hemagglutinin and neuraminidase. For type A influenza virus, there are 16 hemagglutinin subtypes and 9 neuraminidase subtypes, and a particular influenza virus A is identified by its hemagglutinin and neuraminidase subtypes. Thus, an H3N2 influenza A virus has a subtype 3 hemagglutinin and a subtype 2 neuraminidase; an H5N1 influenza A virus has a subtype 5 hemagglutinin and a subtype 1 neuraminidase. The virus uses hemagglutinin and neuraminidase to infect cells. Infection is initiated by the activation of the hemagglutinin by enzymatic cleavage into two fragments, HA1 and HA2; the cleavage results in a significant structural transformation of the molecule (Skehel et al., 2000; Wilson et al., 1990). The biological aggregation state of influenza hemagglutinin is a trimer, while that of the neuraminidase is a tetramer. In the examples that follow, candidate vaccines are designed for two different hemagglutinin and two different neuraminidase subtypes. The uncleaved hemagglutinin molecule is used as the basis for the hemagglutinin vaccines. A survey of the structures of the seven unique antibody-hemagglutinin and antibody-neuraminidase complexes currently available in the Protein Data Bank (Entries: 1KEN, 1EO8, 1QFU, 2VIR, 2AEP, 1NMB, 1NCD) reveals that the epitopes may extend up to 24 Angstroms from the epitope center but that 93.3% of the residues in those epitopes are within a 16-Angstrom radius from the centers. Thus, a 16-Angstrom radius is used in the examples.

### EXAMPLE 1

Design of possible vaccines against human H3 influenza A viruses based on the hemagglutinin molecule:

### Structural and Sequence Data:

Amino-acid sequence data were obtained from the Influenza Sequence Database (ISD) maintained at the Los Alamos National Laboratory (Macken et al., 2001) (http://www.flu.lanl.gov) and from the sequence database maintained at the National Center for Biotechnology Information at the National Library of Medicine (NCBI/NLM) (http://www.ncbi.nlm.nih.gov). Three-dimensional structural data were obtained from the Protein Data Bank.

Experimentally-determined three-dimensional structural data are available for only one uncleaved H3 hemagglutinin molecule.

The three-dimensional structure of the uncleaved, bromelain-released H3 hemagglutinin from the influenza A virus, A/AICHI/68 (with the arginine at the cleavage site replaced by glutamine), has been provided by X-ray crystallography (Chen et al., 2002) (Protein Data Bank entry 1 HA0). Hereinafter, this H3 hemagglutinin will be referred to simply as 1HA0.

The design of possible vaccines against human H3 influenza A viruses currently prevalent in the Philippines based on the hemagglutinin molecule will now be illustrated using the hemagglutinin from the influenza A virus, A/Philippines/1290/2004, whose amino acid sequence is available as entry ISDN110519 in the ISD database. The ISDN110519 entry contains only the HA1 fragment of the molecule (residues 1-329, sequential numbering). A more complete molecule was generated by adding the HA2 fragment of the hemagglutinin from the influenza virus, A/Fujian/411/02-like, available as entry DQ227423 from the NCBI/NLM sequence database. The composite molecule, which corresponds to the bromelain-released fragment of the mature hemagglutinin molecule, includes residues 1-494 (sequential numbering) and is presented as SEQ ID NO: 1, where the segment bounded by positions 298 and 310 had been replaced by SEQ ID NO: 21, an immunodominant T-cell epitope among humans (Hennecke et al., 2000). Hereinafter, this composite H3 hemagglutinin, with the T-cell epitope imposed, will be referred to simply as ISDN110519.

A three-dimensional model for ISDN110519 was generated with the help of the protein modeling server, SWISS-MODEL (Peitsch, 1995; Guex and Peitsch, 1997; Schwede et al., 2003) (http://swissmodel.expasy.org//SWISS-MODEL.html), using the 1HA0 structure as template. In generating the biological aggregation state of the ISDN110519 hemagglutinin, the 1HA0 trimer structure was used as template. All subsequent calculations were based on the ISDN110519 trimer.

### Solvent Accessibilities:

The solvent accessibilities of the individual residues of ISDN110519 were obtained using the program DSSP. Fractional solvent accessibility for each amino acid was estimated by dividing the solvent accessibility obtained from DSSP by the total surface area of the amino acid (obtained from http://prowl.rockefeller.edu/aainfo/volume.htm).

### Calculation of Antigenicities and Identification of the Putative Immunodominant Epitopes:

Antigenicity calculations were performed on the modeled ISDN110519 molecule. The structural parameters, zz1-zz3, provided by Sandberg et al. (1998) (Table 1) were used in the calculation of antigenicities. The antigenicity of a region centered at each alpha-carbon position was computed by taking the sum of the structural parameters corresponding to all the residues within 16 Angstroms of the alpha-carbon. The solvent accessibilities of the individual ISDN110519 residues were used as weighting factors in the calculation of the antigenicities. The average antigenicity value for the molecule and the root-mean-square deviation from the average were computed. Regions with antigenicity values at least two root-mean-square (r.m.s.) deviations above the average were identified as the putative immunodominant epitopes.

### De-Antigenization of the Putative Immunodominant Epitopes:

Residues within each putative immunodominant epitope, with fractional solvent accessibilities of 40 percent or higher and whose contribution to the antigenicity value of the epitope is at least 3 percent, were replaced according to the rules proposed above (Table 1). No residues within the segment bounded by positions 298 and 329 were replaced. The segment, 298-329, includes the immunodominant T-cell epitope at 298-310 and the cleavage site at 321 (Skehel et al., 1975). After the replacements, a new model based on the revised amino-acid sequence was generated as described above and antigenicity calculations were performed on this new model.

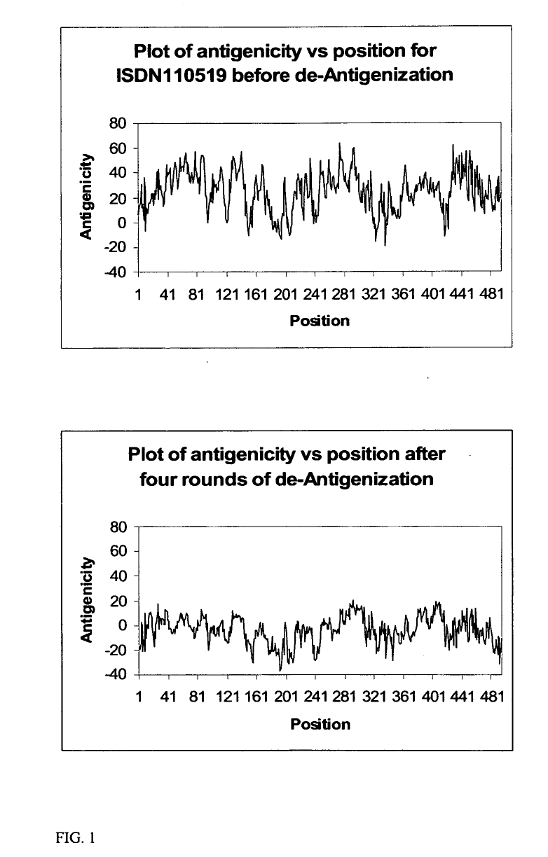

Four rounds of de-Antigenization, i.e. modeling, followed by antigenicity calculations, followed by amino-acid replacements, were computed. The first model of ISDN110519 had an average antigenicity value of 25.3 (r.m.s. deviation=16.0) (arbitrary units). A total of 25 amino-acid replacements were performed, yielding SEQ ID NO: 2. This resulted in an average antigenicity value of 10.8 (r.m.s. deviation=13.1); 17 more changes were suggested, yielding SEQ ID NO: 3. The additional replacements resulted in an average antigenicity value of 1.0 (r.m.s. deviation=13.8); 6 more changes were suggested, yielding SEQ ID NO: 4. This resulted in an average antigenicity value of −2.3 (r.m.s. deviation=12.0); 4 more changes were suggested, yielding SEQ ID NO: 5. This resulted in an average antigenicity value of −3.6 (r.m.s. deviation=11.1); no more changes were suggested after this round. The plots of antigenicities computed for ISDN110519, before and after the four rounds of de-Antigenization, are presented in FIG. 1.

### Possible Vaccines Against Human H3 Influenza A Viruses Currently Prevalent in the Philippines:

Since every round of de-Antigenization of ISDN110519 resulted in a significant decrease in the antigenicity of the putative immunodominant epitopes, any of the derivative amino-acid sequences (SEQ ID NO: 2 through 5), or a polynucleotide derived from it, could be the basis of a molecule useful for immunization, for example, as a vaccine for humans, or for the generation of therapeutic antibodies, against human H3 influenza A viruses currently prevalent in the Philippines. The best candidate is probably the one represented by the sequence after the fourth round of de-Antigenization (SEQ ID NO: 5).

### EXAMPLE 2

Design of possible vaccines for humans against N2 influenza A viruses based on the neuraminidase molecule:

### Structural and Sequence Data:

A crystallographically-determined structure for the pronase-released fragment of an N2 neuraminidase from influenza A virus is available from the Protein Data Bank. The neuraminidase is from the influenza virus, A/Tokyo/3/67 (Varghese et al., 1991) (Entry 2BAT), and hereinafter is simply referred to as 2BAT.

The design of possible vaccines for humans against N2 influenza A viruses based on the neuraminidase molecule will now be illustrated using the amino acid sequence of the neuraminidase from the influenza A virus, A/California/7/2004 (egg-passaged), available as entry ISDN117767 in the ISD database. This virus is one of those currently prevalent in the Philippines. Hereinafter, this N2 neuraminidase will be referred to simply as ISDN117767.

The segment of ISDN117767, which corresponds to the pronase-released fragment of the mature neuraminidase molecule, includes residues 1-388 (sequential numbering) and is presented as SEQ ID NO: 6. A three-dimensional model for ISDN117767 was generated with the help of the protein modeling server, SWISS-MODEL, using the 2BAT structure as template. In generating the biological aggregation state of the ISDN117767 neuraminidase, the 2BAT tetramer structure was used as template. All subsequent calculations were based on the ISDN117767 tetramer.

### Solvent Accessibilities:

The solvent accessibilities of the individual residues of ISDN117767 were obtained using the program DSSP. Fractional solvent accessibility for each amino acid was estimated by dividing the solvent accessibility obtained from DSSP by the total surface area of the amino acid (obtained from http://prowl.rockefeller.edu/aainfo/volume.htm).

### Calculation of Antigenicities and Identification of the Putative Immunodominant Epitopes:

Antigenicity calculations were performed on the modeled ISDN117767 molecule. The structural parameters, zz1-zz3, provided by Sandberg et al. (1998) (Table 1) were used in the calculation of antigenicities. The antigenicity of a region centered at each alpha-carbon position was computed by taking the sum of the structural parameters corresponding to all the residues within 16 Angstroms of the alpha-carbon. The solvent accessibilities of the individual ISDN117767 residues were used as weighting factors in the calculation of the antigenicities. The average antigenicity value for the molecule and the root-mean-square deviation from the average were computed. Regions with antigenicity values at least two root-mean-square (r.m.s.) deviations above the average were identified as the putative immunodominant epitopes.

### De-Antigenization of the Putative Immunodominant Epitopes:

Residues within each putative immunodominant epitope, with fractional solvent accessibilities of 40 percent or higher and whose contribution to the antigenicity value of the epitope is at least 3 percent, were replaced according to the rules proposed above (Table 1). No residues within the segment bounded by positions 131 and 146 were replaced. The segment, 131-146, is identified as a possible T-cell epitope by the program SYFPEITHI. After the replacements, a new model based on the revised amino-acid sequence was generated as described above and antigenicity calculations were performed on this new model.

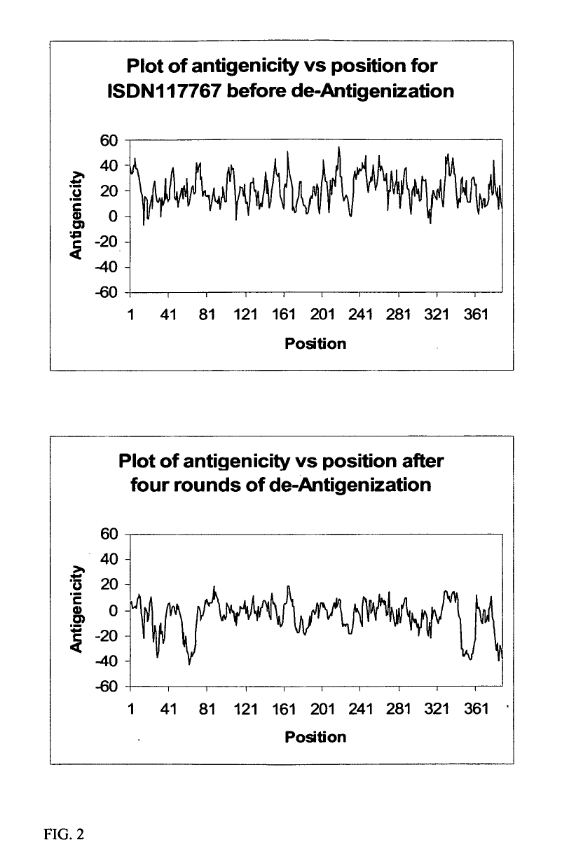

Four rounds of de-Antigenization were computed. The first model of ISDN117767 had an average antigenicity value of 21.0 (r.m.s. deviation=11.5) (arbitrary units). A total of 25 amino-acid replacements were performed, yielding SEQ ID NO: 7. This resulted in an average antigenicity value of 1.2 (r.m.s. deviation=12.0); 3 more changes were suggested, yielding SEQ ID NO: 8. The additional replacements resulted in an average antigenicity value of −0.2 (r.m.s. deviation=11.5); 2 more changes were suggested, yielding SEQ ID NO: 9. This resulted in an average antigenicity value of −1.4 (r.m.s. deviation=11.2); 4 more changes were suggested, yielding SEQ ID NO: 10. This resulted in an average antigenicity value of −4.4 (r.m.s. deviation=12.6); no more changes were suggested after this round. The plots of antigenicities computed for ISDN117767, before and after the four rounds of de-Antigenization, are presented in FIG. 2.

### Possible Vaccines Against N2 Influenza A Viruses Currently Prevalent in the Philippines:

Since every round of de-Antigenization of ISDN117767 resulted in a significant decrease in the antigenicity of the putative immunodominant epitopes, any of the derivative amino-acid sequences (SEQ ID NO: 7 through 10), or a polynucleotide derived from it, could be the basis of a molecule useful for immunization, for example, as a vaccine for humans, or for the generation of therapeutic antibodies, against N2 influenza A viruses currently prevalent in the Philippines. The best candidate is probably the one represented by the sequence after the fourth round of de-Antigenization (SEQ ID NO: 10).

### EXAMPLE 3

Design of possible vaccines for humans against avian-derived, pathogenic H5 influenza A viruses based on the hemagglutinin molecule:

### Structural and Sequence Data:

Experimentally-determined three-dimensional structural data are available for two H5 hemagglutinin molecules.

The three-dimensional structure of the bromelain-released H5 hemagglutinin from the avian influenza A virus, A/Duck/Singapore/3/97, has been provided by X-ray crystallography (Ha et al., 2002) (Protein Data Bank entry 1JSM). Hereinafter, this H5 hemagglutinin will be referred to simply as 1JSM. The 1JSM structure is for a molecule that had been cleaved into the HA1 and HA2 subunits.

The three-dimensional structure of the bromelain-released H5 hemagglutinin from the avian influenza A virus, A/VIET NAM/1203/2004, has been provided by X-ray crystallography (Stevens et al., 2006) (Protein Data Bank entry 2FK0). Hereinafter, this H5 hemagglutinin will be referred to simply as 2FK0. The 2FK0 structure is for a molecule that had been cleaved into the HA1 and HA2 subunits also.

No three-dimensional structure is available for an intact H5 hemagglutinin molecule. Three-dimensional structures are available for an intact H3 hemagglutinin (Protein Data Bank entry lHAO (Chen et al., 1998)) and for an intact HI hemagglutinin (Protein Data Bank entry 1RD8 (Stevens et al., 2004)).

The design of possible vaccines against avian-derived, pathogenic H5 influenza A viruses based on the hemagglutinin molecule will now be illustrated using the hemagglutinin from the avian-derived, pathogenic influenza A virus, A/Thailand/3(SP-83)/2004 (Puthavathana et al., 2005), which had been isolated from a human victim and whose amino acid sequence is available as entry AAS89004 in the NCBI/NLM database.

The segment of AAS89004, which corresponds to the bromelain-released fragment of the mature hemagglutinin molecule, includes residues 1-505 (sequential numbering) and is presented as SEQ ID NO: 11, where the segment bounded by positions 303 and 315 had been replaced by SEQ ID NO: 21, an immunodominant T-cell epitope among humans (Hennecke et al., 2000). Hereinafter, this composite H5 hemagglutinin will be referred to simply as AAS89004. A three-dimensional model for AAS89004 was generated with the help of the protein modeling server, SWISS-MODEL, using the 1JSM, 2FK0, and 1RD8 structures as templates. In generating the biological aggregation state of the AAS89004 hemagglutinin, the 1RD8 trimer structure was used as template. All subsequent calculations were based on the AAS89004 trimer.

### Solvent Accessibilities:

The solvent accessibilities of the individual residues of AAS89004 were obtained using the program DSSP. Fractional solvent accessibility for each amino acid was estimated by dividing the solvent accessibility obtained from DSSP by the total surface area of the amino acid (obtained from http://prowl.rockefeller.edu/aainfo/volume.htm).

### Calculation of Antigenicities and Identification of the Putative Immunodominant Epitopes:

Antigenicity calculations were performed on the modeled AAS89004 molecule. The structural parameters, zz1-zz3, provided by Sandberg et al. (1998) (Table 1) were used in the calculation of antigenicities. The antigenicity of a region centered at each alpha-carbon position was computed by taking the sum of the structural parameters corresponding to all the residues within 16 Angstroms of the alpha-carbon. The solvent accessibilities of the individual AAS89004 residues were used as weighting factors in the calculation of the antigenicities. The average antigenicity value for the molecule and the root-mean-square deviation from the average were computed. Regions with antigenicity values at least two root-mean-square (r.m.s.) deviations above the average were identified as the putative immunodominant epitopes.

### De-Antigenization of the Putative Immunodominant Epitopes:

Residues within each putative immunodominant epitope, with fractional solvent accessibilities of 40 percent or higher and whose contribution to the antigenicity value of the epitope is at least 3 percent, were replaced according to the rules proposed above (Table 1). Since the candidate vaccines being designed here are intended for humans, no residues within the segment bounded by positions 303 and 315, the immunodominant human T-cell epitope (Hennecke et al., 2000), were replaced. Further, no residues within the probable cleavage site, 325-330, for the hemagglutinin of highly virulent H5 avian influenza viruses (Horimoto, T. et al., 1994), were replaced; antibodies to the cleavage site have been shown to be able to inhibit activation of the hemagglutinin molecule (Bianchi et al., 2005; Horvath et al., 1998). In all, no residues within the segment, 303-338, were replaced during the de-Antigenization of AAS89004. After the replacements, a new model based on the revised amino-acid sequence was generated as described above and antigenicity calculations were performed on this new model.

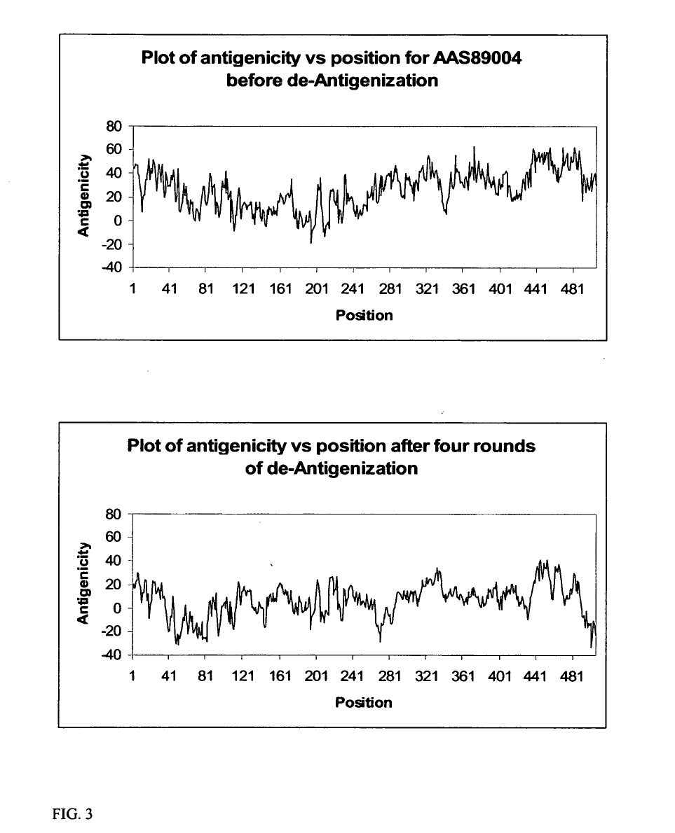

Four rounds of de-Antigenization were computed. The first model of AAS89004 had an average antigenicity value of 26.4 (r.m.s. deviation=16.1) (arbitrary units). A total of 17 amino-acid replacements were performed, yielding SEQ ID NO: 12. This resulted in an average antigenicity value of 17.6 (r.m.s. deviation=12.6); 16 more changes were suggested, yielding SEQ ID NO: 13. The additional replacements resulted in an average antigenicity value of 8.7 (r.m.s. deviation=14.7); 3 more changes were suggested, yielding SEQ ID NO: 14. This resulted in an average antigenicity value of 6.7 (r.m.s. deviation=14.1); one more change was suggested, yielding SEQ ID NO: 15. This resulted in an average antigenicity value of 6.3 (r.m.s. deviation=14.0); no more changes were suggested after this round. The plots of antigenicities computed for AAS89004, before and after the four rounds of de-Antigenization, are presented in FIG. 3.

### Possible Vaccines Against Pathogenic H5 Influenza A Viruses:

Since every round of de-Antigenization of AAS89004 resulted in a significant decrease in the antigenicity of the putative immunodominant epitopes, any of the derivative amino-acid sequences (SEQ ID NO: 12 through 15), or a polynucleotide derived from it, could be the basis of a molecule useful for immunization, for example, as a vaccine for humans, or for the generation of therapeutic antibodies, against avian-derived, pathogenic H5 influenza A viruses that were prevalent in Thailand in 2004 and which are probably still circulating in that country, as well as similar viruses in other countries. The best candidate is probably the one represented by the sequence after the fourth round of de-Antigenization (SEQ ID NO: 15).

### EXAMPLE 4

Design of possible vaccines for humans against avian-derived, pathogenic N1 influenza A viruses based on the neuraminidase molecule:

### Structural and Sequence Data:

Experimentally-determined three-dimensional structural data are available for only two neuraminidase molecules.

A crystallographically-determined structure of the pronase-released fragment of the N2 neuraminidase from the influenza virus, A/Tokyo/3/67, (Varghese et al., 1991) is available from the Protein Data Bank (Entry 2BAT), hereinafter referred to simply as 2BAT.

A crystallographically-determined structure of the pronase-released fragment of the N9 neuraminidase from the influenza virus, A/Tern/Australia/G70C/75, (Varghese et al.,) is available from the Protein Data Bank (Entry 7NN9), hereinafter referred to simply as 7NN9.

No three-dimensional structure is available for an N1 neuraminidase molecule.

The design of possible vaccines for humans against avian-derived, pathogenic N1 influenza A viruses based on the neuraminidase molecule will now be illustrated using the neuraminidase from the avian-derived, pathogenic human influenza A virus, A/Thailand/3(SP-83)/2004 (Puthavathana et al., 2005), which was isolated from a human victim and whose amino acid sequence is available as entry AAS89005 in the NCBI/NLM database. Hereinafter, this N, 1 neuraminidase will be referred to simply as AAS89005.

The segment of AAS89005, which corresponds to the pronase-released fragment of the mature neuraminidase molecule, includes residues 1-386 (sequential numbering) and is presented as SEQ ID NO: 16. A three-dimensional model for AAS89005 was generated with the help of the protein modeling server, SWISS-MODEL, using the 1F8E structure as template. In generating the biological aggregation state of the AAS89005 neuraminidase, the 1F8E tetramer structure was used as template. All subsequent calculations were based on the AAS89005 tetramer.

### Solvent Accessibilities:

The solvent accessibilities of the individual residues of AAS89005 were obtained using the program DSSP. Fractional solvent accessibility for each amino acid was estimated by dividing the solvent accessibility obtained from DSSP by the total surface area of the amino acid (obtained from http://prowl.rockefeller.edu/aainfo/volume.htm).

### Calculation of Antigenicities and Identification of the Putative Immunodominant Epitopes:

Antigenicity calculations were performed on the modeled AAS89005 molecule. The structural parameters, zz1-zz3, provided by Sandberg et al. (1998) (Table 1) were used in the calculation of antigenicities. The antigenicity of a region centered at each alpha-carbon position was computed by taking the sum of the structural parameters corresponding to all the residues within 16 Angstroms of the alpha-carbon. The solvent accessibilities of the individual AAS89005 residues were used as weighting factors in the calculation of the antigenicities. The average antigenicity value for the molecule and the root-mean-square deviation from the average were computed. Regions with antigenicity values at least two root-mean-square (r.m.s.) deviations above the average were identified as the putative immunodominant epitopes.

### De-Antigenization of the Putative Immunodominant Epitopes:

Residues within each putative immunodominant epitope, with fractional solvent accessibilities of 40 percent or higher and whose contribution to the antigenicity value of the epitope is at least 3 percent, were replaced according to the rules proposed above (Table 1). No residues within the segments bounded by positions 120 and 134, and by positions 339 and 353, were replaced. The segments, 120-134 and 339-353, are identified as possible T-cell epitopes by the program SYFPEITHI. After the replacements, a new model based on the revised amino-acid sequence was generated as described above and antigenicity calculations were performed on this new model.

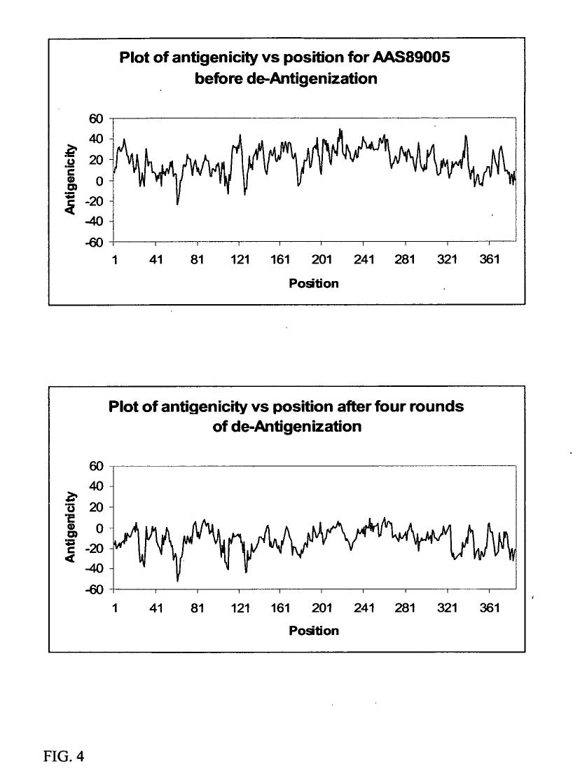

Four rounds of de-Antigenization, i.e. modeling, followed by antigenicity calculations, followed by amino-acid replacements, were computed. The first model of AAS89005 had an average antigenicity value of 18.2 (r.m.s. deviation=12.7) (arbitrary units). A total of 15 amino-acid replacements were performed, yielding SEQ ID NO: 17. This resulted in an average antigenicity value of 5.5 (r.m.s. deviation=10.4); 14 more changes were suggested, yielding SEQ ID NO: 18. The additional replacements resulted in an average antigenicity value of −6.4 (r.m.s. deviation=10.8); 7 more changes were suggested, yielding SEQ ID NO: 19. This resulted in an average antigenicity value of −9.6 (r.m.s. deviation=10.1); 3 more changes were suggested, yielding SEQ ID NO: 20. This resulted in an average antigenicity value of −11.7 (r.m.s. deviation=10.8); no more changes were suggested after this round. The plots of antigenicities computed for AAS89005, before and after the four rounds of de-Antigenization, are presented in FIG. 4.

### Possible Vaccines Against Avian-Derived, Pathogenic N1 Influenza A Viruses:

Since every round of de-Antigenization of AAS89005 resulted in a significant decrease in the antigenicity of the putative immunodominant epitopes, any of the derivative amino-acid sequences (SEQ ID NO: 17 through 20), or a polynucleotide derived from it, could be the basis of a molecule useful for immunization, for example, as a vaccine for humans, or for the generation of therapeutic antibodies, against avian-derived, pathogenic N, 1 influenza A viruses that were prevalent in Thailand in 2004 and which are probably still circulating in that country, as well as similar viruses in other countries. The best candidate is probably the one represented by the sequence after the fourth round of de-Antigenization (SEQ ID NO: 20).

TABLE 1
The amino acid parameters used in the calculation of antigenicities and the replacement suggestions

| | | | | | | | | | | | If in | | | |
|---|---|---|---|---|---|---|---|---|---|---|---|---|---|---|
| Amino | | | | | | | Helix | Sheet | Coil | Turn | Helix | Sheet | Coil | Turn |
| acid | zz1 | zz2 | zz3 | zz4 | zz5 | SDGly | Propensities | | | | Change to: | | | |
| Ala | 0.24 | −2.32 | 0.60 | −0.14 | 1.30 | 60.0 | 0.00 | 0.47 | −0.26154 | 0.83 | — | — | — | — |
| Arg | 3.52 | 2.50 | −3.50 | 1.99 | −0.17 | 125.0 | 0.21 | 0.35 | −0.17659 | 0.82 | Ala | Thr | Ala | Ala |
| Asn | 3.05 | 1.62 | 1.04 | −1.15 | 1.61 | 80.0 | 0.65 | 0.40 | 0.22989 | 1.44 | Ala | Thr | Ser | Gly |
| Asp | 3.98 | 0.93 | 1.93 | −2.46 | 0.75 | 94.0 | 0.69 | 0.72 | 0.22763 | 1.41 | Ala | Thr | Ser | Gly |
| Cys | 0.84 | −1.67 | 3.71 | 0.18 | −2.65 | 159.0 | 0.68 | 0.25 | −0.015152 | 1.08 | — | — | — | — |
| Gln | 1.75 | 0.50 | −1.44 | −1.34 | 0.66 | 87.0 | 0.39 | 0.34 | −0.187677 | 0.94 | Ala | Thr | Ala | Thr |
| Glu | 3.11 | 0.26 | −0.11 | −3.04 | −0.25 | 98.0 | 0.40 | 0.35 | −0.20469 | 1.01 | Ala | Thr | Ala | Thr |
| Gly | 2.05 | −4.06 | 0.36 | −0.82 | −0.38 | 0.0 | 1.00 | — | 0.43323 | 1.48 | — | — | — | — |
| His | 2.47 | 1.95 | 0.26 | 3.90 | 0.09 | 98.0 | 0.56 | 0.37 | −0.0012174 | 1.07 | Ala | Thr | Thr | Thr |
| Ile | −3.89 | −1.73 | −1.71 | −0.84 | 0.26 | 135.0 | 0.41 | 0.10 | −0.42224 | 0.59 | — | — | — | — |
| Leu | −4.28 | −1.30 | −1.49 | −0.72 | 0.84 | 138.0 | 0.21 | 0.32 | −0.33793 | 0.66 | — | — | — | — |
| Lys | 2.29 | 0.89 | −2.49 | 1.49 | 0.31 | 127.0 | 0.26 | 0.34 | −0.100092 | 1.01 | Ala | Thr | Thr | Thr |
| Met | −2.85 | −0.22 | 0.47 | 1.94 | −0.98 | 127.0 | 0.24 | 0.26 | −0.22590 | 0.57 | — | — | — | — |
| Phe | −4.22 | 1.94 | 1.06 | 0.54 | −0.62 | 153.0 | 0.54 | 0.13 | −0.22557 | 0.89 | Ala | Thr | Ala | Ala |
| Pro | −1.66 | 0.27 | 1.84 | 0.70 | 2.00 | 42.0 | 3.01 | — | 0.55232 | 1.38 | — | — | — | — |
| Ser | 2.39 | −1.07 | 1.15 | −1.39 | 0.67 | 56.0 | 0.50 | 0.30 | 0.14288 | 1.15 | — | — | — | — |
| Thr | 0.75 | −2.18 | −1.12 | −1.46 | −0.40 | 59.0 | 0.66 | 0.06 | 0.0088780 | 1.00 | — | — | — | — |
| Trp | −4.36 | 3.94 | 0.59 | 3.44 | −1.59 | 184.0 | 0.49 | 0.24 | −0.243375 | 0.70 | Ala | Thr | Ala | Val |
| Tyr | −2.54 | 2.44 | 0.43 | 0.04 | −1.47 | 147.0 | 0.53 | 0.11 | −0.20751 | 0.92 | Ala | Thr | Ala | Thr |
| Val | −2.59 | −2.64 | −1.54 | −0.85 | −0.02 | 109.0 | 0.61 | 0.13 | −0.38618 | 0.70 | — | — | — | — |

Footnote to Table 1:
The zz values are from Sandberg et al. (1998). The SDGly values are from Grantham (1974) and represent the structural dissimilarities of the various amino acids relative to glycine. The helix propensities are from Pace et al. (1998). The beta sheet propensities are from Street et al. (1999). The coil propensities are from Linding et al. (2003). The turn propensities are from Hutchinson et al. (1994). A dash in the replacement suggestions signifies that no change is recommended.

What are claimed are:

1. A method for reducing the antigenicity of putative immunodominant epitopes in a protein antigen of a particular pathogen, the method comprising:
a) identifying the putative immunodominant epitopes of the antigen and the amino acid residues which constitute those epitopes; and
b) replacing the residues, which contribute the most to the antigenicity of the putative immunodominant epitopes, with amino acids whose physicochemical properties will effectively reduce the antigenicity of those epitopes while preserving structure.

2. A polypeptide designed using claim 1.

3. A polynucleotide derived from a polypeptide of claim 2.

4. A pharmaceutical composition comprising the polypeptide of claim 2 and a pharmaceutically acceptable carrier.

5. A pharmaceutical composition comprising the polynucleotide of claim 3 and a pharmaceutically acceptable carrier.

6. A pharmaceutical composition of claim 4 that is used for immunization, for example, as a vaccine, or for the generation of therapeutic antibodies.

7. A pharmaceutical composition of claim 5 that is used for immunization, for example, as a vaccine, or for the generation of therapeutic antibodies.

8. A method for reducing the antigenicity of putative immunodominant epitopes in a protein antigen of a particular pathogen that is based on the method described in claim 1.

9. A polypeptide designed using a method described in claim 8.

10. A polynucleotide derived from a polypeptide of claim 9.

11. A pharmaceutical composition comprising the polypeptide of claim 8 and a pharmaceutically acceptable carrier.

12. A pharmaceutical composition comprising the polynucleotide of claim 10 and a pharmaceutically acceptable carrier.

16. A pharmaceutical composition of claim 11 that is used for immunization, for example, as a vaccine, or for the generation of therapeutic antibodies.

17. A pharmaceutical composition of claim 12 that is used for immunization, for example, as a vaccine, or for the generation of therapeutic antibodies.
